(19) United States
(12) Patent Application Publication
(10) Pub. No.: US 2009/0182229 A1
(43) Pub. Date: Jul. 16, 2009

(54) UltraSound System With Highly Integrated ASIC Architecture

(76) Inventor: Robert Gideon Wodnicki, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/972,011

(22) Filed: Jan. 10, 2008

(51) Int. Cl. A61B 8/00

(52) U.S. Cl. 600/437

(57) ABSTRACT

An ultrasound system and method of configuring an ultrasound system. In one embodiment, the system includes an array of transducer cells grouped in acoustical subelements. Transducer cells in different acoustical subelements are operable according to different pulser timing signals. An integrated circuit structure includes an array of circuit support cells. A first of the support cells provides circuitry for implementing selectable timing signals and high voltage pulse generation to propagate acoustic signals from multiple transducer cells. Connective paths extend between the transducer cells and the first of the circuit support cells to effect generation of acoustic signals and receipt of echo data. A first of the paths extends between a first transducer cell positioned in a first of the different ones of the acoustical subelements and circuitry in the first of the support cells, and a second of the paths extends between a second transducer cell positioned in a second of the different ones of the acoustical subelements and circuitry in the first of the support cells.

### STATEMENT REGARDING FEDERALLY SPONSORED RESEARCH AND DEVELOPMENT

The United States Government may have certain rights in this invention pursuant to U.S. Government Contract Number 1R01 EB002485 awarded by the National Institutes of Health.

### BACKGROUND OF THE INVENTION

This application is related to 206174-4 filed January 2008. The invention generally relates to ultrasound systems of the type which incorporate application specific integrated circuits (ASICs) for use in conjunction with a two dimensional array of transducer elements. In particular, the invention includes embodiments wherein both low-voltage and high-voltage circuits are integrated within individual circuit cells of an ASIC and the ASIC is located adjacent the array of transducer elements. In such applications the probe handle is typically connected via cabling to a portable unit or a console which provides image processing and control circuitry.

Image resolution is partly a function of the number of transducer elements that respectively constitute the transmit and receive apertures of the transducer array. Accordingly, to achieve high image quality, a large number of transducer elements is desirable for both two- and three-dimensional imaging applications. For medical imaging applications the ultrasonic transducer elements are normally located in a hand-held transducer probe unit connected to an electronics unit that processes the transducer signals and generates ultrasound images. The transducer probe may contain both ultrasound transmit circuitry and ultrasound receive circuitry. The electronics unit may be a light-weight portable system or may be a large console depending on the performance capability of the system. One function of the electronics unit connected to the transducer probe is to provide imaging and control functions for transmitting and receiving acoustic signals. Such systems incorporate switching circuitry to program and control the generation of acoustic patterns and to process the echo data into images.

Ultrasound systems are of growing complexity, particularly in the field of medical diagnostics because of the desire to improve system performance, e.g., image resolution, while maintaining or reducing the size and weight of the probe unit. Advancements in the field are also of importance to applications concerning non-destructive evaluation of materials, such as for examining the integrity of castings, forgings and extrusions, including pipelines, solid fuel and components of mechanical systems.

For the purposes of this disclosure, “low voltage” means any voltage level that is readily implemented in widely available “standard” semiconductor processes. This could be anywhere from less than 2.5 V to 5 V (for CMOS) up to 25 to 30 V (for BiCMOS). In contrast, “high voltage” means voltage levels that are only accessible if more specialized semiconductor processes and device structures are used (e.g., DMOSFETs, silicon on insulator (SOI), trench isolation, etc.) Therefore any voltage level from about 30 V up to as high as 500 V or more may be considered to be “high voltage”. As used herein, low voltage and high voltage correspond to relative ranges which in some applications may overlap with one another but in the context of ultrasound systems will be understood to relate to different types of circuit functions, e.g., low voltage logic and high voltage pulse generation to drive acoustic transducers. In a conventional ultrasound imaging system an array of transducer cells alternately transmits an ultrasound beam and then receives the reflected beam, or echo, from an object or patient under study. Such scanning comprises a series of measurements in which one or more focused ultrasonic waves are transmitted while the system is in a transmission mode. Through switching circuitry the system is then transitioned into a receive mode wherein the reflected ultrasonic waves are converted into electrical signals which are beamformed and processed for display. With timing and switching circuitry the reflected signals may be processed to provide image information along a succession of ranges over which the received ultrasonic waves propagate.

Semiconductor processes are used to manufacture ultrasonic transducer cells known as micromachined ultrasonic transducers (MUTs). These may be of the capacitive (cMUT) variety or the piezoelectric (PMUT) variety. MUTs in general are tiny diaphragm-like devices with electrodes that are modulated with a high voltage signal generated by pulser circuitry to vibrate the diaphragm of the device and thereby transmit a sound wave. Alternately, the MUTs and associated circuitry can be placed in the receive mode to obtain a reflected portion of the transmitted sound which is first converted into vibration signals in the diaphragm. The vibrations are converted into a modulated capacitance. Signals from multiple MUT's are coprocessed to develop images.

MUTs can be manufactured using semiconductor fabrication processes, such as microfabrication processes grouped under the heading “micromachining”. The systems resulting from such micromachining processes are typically referred to as “micromachined electro-mechanical systems” (MEMS). As explained in U.S. Pat. No. 6,359,367, micromachining is the formation of microscopic structures using a combination or subset of (A) Patterning tools (generally lithography such as projection-aligners or wafer-steppers), and (B) Deposition tools such as PVD (physical vapor deposition), CVD (chemical vapor deposition), LPCVD (low-pressure chemical vapor deposition), PECVD (plasma chemical vapor deposition), and (C) Etching tools such as wet-chemical etching, plasma-etching, ion-milling, sputter-etching or laser-etching.

For high resolution ultrasound imaging it is desirable that the array of transducer cells be two dimensional. A driving voltage is typically applied among different groups of transducer cells or subelements of transducer cells in the array. A defined group of transducer cells for acoustic wave generation or reception may be referred to as an element. With variation in the amplitude and phase of transducer cells in different groups, desired ultrasonic wave patterns are generated. In this manner it is possible to sequentially select portions of a region for image generation and it is possible to vary the depth of focus in order to image different portions of a region under examination. Also, when the transducer cells are placed in the receive mode to process reflected sound, the voltages produced in the transducer cells can be processed in accord with a predetermined timing pattern so that a composite generated from the signals corresponds to a particular focal zone.

Acoustic transmission is achieved in part by propagating multiple channels of timing pulses and selecting certain ones of these to control the transmitters. Pulses in different channels have relative delay or phase characteristics with respect to one another. This arrangement can be implemented with a digital scanning architecture wherein groups of elements can be defined or re-defined at any given time by selecting groups of transducer cells to operate under the control of timing pulses carried in a particular channel. The relative time delay among individual channels may be varied, and the connection of channels to define groups of transducer cells may be varied in order to achieve real-time flexibility to generate a variety of images, e.g., according to differing depths of focus. Imaging of received signals is accomplished in a similar way.

High-voltage transmit circuitry is used to drive the individual ultrasonic transducer cells. Low-voltage, high-density digital logic circuitry is used to provide timing signals to gate the high-voltage drivers in either the transmit or the receive mode. The high-voltage drivers typically operate at voltages of up to approximately +/−100 volts. The low-voltage logic circuitry may have an operating voltage on the order of 5 volts in the case of TTL logic. The high-voltage drivers may be fabricated as discrete components or as integrated circuits, while the low-voltage logic circuitry may be fabricated as a separate integrated circuit or combined with the high-voltage circuitry on a single chip. In addition to transmit circuitry including the high-voltage drivers and low-voltage logic circuitry, the transducer head may include low-noise, low-voltage analog receive circuitry. The low-voltage receive circuitry, like the transmit logic circuitry, typically has an operating voltage on the order of 5 volts, and may be a separate integrated circuit or may be fabricated with the low-voltage transmit logic circuitry as a monolithic integrated circuit.

Typically, a transmit/receive switch is placed between the output-stage transistors of the high voltage transmit circuitry and the transducer element. The transmit/receive switch is also connected to the low-voltage receive circuit. The transmit/receive switch has two states. In the transmit state, the transmit/receive switch connects the output-stage transistors to the ultrasonic transducer element, while isolating the receive circuit from the high-voltage transmit pulse. In the receive state, the transmit/receive switch isolates the high voltage output-stage transistors from the ultrasonic transducer element and instead connects the receive circuit to the transducer element.

The two-dimensional transducer arrays required for high resolution and three-dimensional imaging typically employ arrays of transducer cells wherein transducer cells adjacent one another are hard-wired into clusters referred to as acoustical subelements. Thousands of acoustical subelements may be configured into an element. For proper beamforming, each of the transducer cells in each element must be connected to an appropriate channel for receiving a high voltage transmission pulse according to a desired timing sequence. Connecting several thousand transducer subelements to receive these driving signals from the correct channels can present technical difficulties due to the number of wire leads that must extend between the transducer array and the timing and control circuitry. This challenge results both from the increased number of transducer elements needed to provide the desired resolution and from the requirements for constructing multi-dimensional images. Even with the integration of the pulser circuits, which form the transducer cell driving signals, into the ASIC cells in the probe unit, the size and weight of the required wiring is a limiting factor. It is desirable to reduce the size and weight of the probe unit and of portable systems generally.

Recently some advancements have been made to further improve performance in ultrasound systems formed with large transducer arrays having integrated pulser circuitry or more optimized switching configurations for reconfigurable arrays. See, for example, Ser. No. 11/172,599 filed Jun. 29, 2005 and Ser. No. 10/978,175 filed Oct. 29, 2004, each now incorporated herein by reference. Still, in order to further improve performance of systems formed with ultrasound transducers in a two-dimensional array configuration, there remains a need to reduce the size and weight of the electronics. This would enable further integration of circuitry such as timing and control electronics into the probe unit while still meeting ergonomic criteria.

Reference will now be made to the drawings in which similar elements in different drawings bear the same reference numerals.

### BRIEF DESCRIPTION OF THE INVENTION

In one form of the invention, an ultrasound system includes a two-dimensional array of ultrasound transducer cells grouped in a plurality of acoustical subelements. The transducer cells in an acoustical subelement are operable together according to a common pulser timing signal, and transducer cells in different acoustical subelements are operable according to different pulser timing signals. An integrated circuit structure includes an array of circuit support cells formed along a plane spaced apart from the array of ultrasound transducer cells. A first of the support cells provides circuitry for implementing selectable timing signals and high voltage pulse generation to propagate acoustic signals from multiple transducer cells, including transducer cells positioned in different ones of the acoustical subelements. A plurality of connective paths extend between the transducer cells and the first of the circuit support cells to effect generation of acoustic signals from the transducer cells and receipt into the first of the circuit support cells of echo data from the multiple transducer cells. A first of the paths extends between a first transducer cell positioned in a first of the different ones of the acoustical subelements and circuitry in the first of the support cells, and a second of the paths extends between a second transducer cell positioned in a second of the different ones of the acoustical subelements and circuitry in the first of the support cells. A plurality of connective paths extends between the first circuit support cell and processing and control circuitry external to the integrated circuit structure to effect operation and signal processing functions in association with the multiple transducer cells.

Other aspects of the invention are disclosed and claimed below.

### BRIEF DESCRIPTION OF THE DRAWINGS

FIG. 1 illustrates an acoustical subelement formed according to an exemplary embodiment wherein seven hexagonal-shaped MUT transducer cells are electrically connected to one another;

FIG. 2 describes a functional circuit arrangement according to which a switching network might work for a particular prior art architecture as well as for a layout architecture in accord with the present invention;

FIG. 3 illustrates an ultrasound system according to an embodiment of the invention;

FIG. 4 illustrates a portion of the support circuitry associated with a subelement according as described for prior art designs;

FIG. 5A is a schematic view of a portion of an array of ASIC cells according to one embodiment of the invention;

FIG. 5B is a simplified plan view of one of the ASIC cells shown in FIG. 5A

FIG. 5C is a simplified partial cross sectional view of the array shown in FIG. 5A, illustrating acoustical subelements and associated ASIC cells;

FIGS. 6A and 6B illustrate exemplary arrangements for routing of I/O lines into an array of ASIC cells;

FIG. 7A illustrates an exemplary column of ASIC cells 92 according to an embodiment of the invention;

FIG. 7B illustrates an exemplary routing of channel lines into ASIC cells according to an embodiment of the invention;

FIG. 8 illustrates a grouped cell which provides support circuitry for multiple acoustical subelements according to the invention;

FIGS. 9A and 9B illustrate a subelement array according to another embodiment of the invention;

FIGS. 10A and 10B illustrate, respectively, a plan view and a cross sectional view of another embodiment of a grouped cell according to the invention; and

FIGS. 11A and 11B illustrate two exemplary arrangements for routing of channel lines according to different embodiments of the invention wherein timing channel select switches are integrated into an array of ASIC cells.

### DETAILED DESCRIPTION OF THE INVENTION

For purposes of illustration, various embodiments of the invention will be described in the context of an array 5 comprising capacitive micromachined ultrasonic transducers (cMUTs). However, it should be understood that the present invention is not limited in application to cMUT arrays, but rather may also be practiced in arrays that employ pMUTs or PZT elements or other transducers found suitable for ultrasound applications.

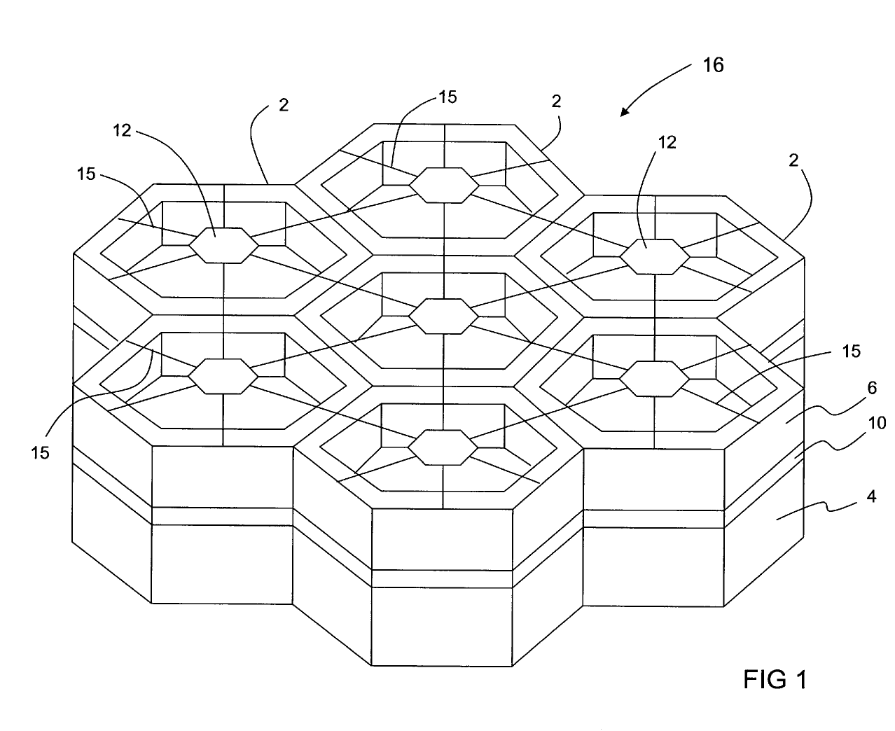

Referring to FIG. 1, a group of cMUT transducer cells 2 is schematically shown. A large array of such cMUT transducer cells 2 is typically fabricated on a substrate 4, such as a heavily doped silicon semiconductive wafer. For each cMUT transducer cell 2, a thin membrane or diaphragm (not shown), which may be made of silicon nitride, is suspended above the substrate, being supported along its periphery by an insulative support 6, typically formed of silicon oxide or silicon nitride. The resulting cavity formed between the membrane and the substrate 4 may be air- or gas-filled or wholly or partially evacuated.

Typically, cMUTs are evacuated as completely as the processes allow. A film or layer of conductive material, such as an aluminum alloy or other suitable conductive material is patterned to form an electrode 12 on each of the membranes, and another film or layer made of conductive material forms a continuous bottom electrode 10 on the substrate 4. The electrode 10 may provide a common ground among all of the transducer cells 2. In other embodiments, a bottom electrode can be formed by appropriate doping of the semiconductive substrate 4. The material patterned to form the electrodes 12 is also patterned to form conductors 15 which extend between electrodes of adjacent cMUT cells 2 and electrically tie selected ones of the electrodes together. In the example of FIG. 1, a cluster of seven cMUT cells 2 is defined by interconnections formed with the conductors 15.

The resulting acoustical subelement 16 comprises a centrally positioned cell 2 surrounded by a ring of six other cells 2. The top electrodes 12 of each cMUT cell 2 in the acoustical subelement 16 are interconnected so that the seven adjoining cells 2 are electrically coupled together by connections that are not switchably disconnectable. In the case of the illustrated cluster of seven hexagonal cells 2, six conductors 15 radiate outward from the top electrodes 12 like “spokes”. In the acoustical subelement 16 the conductors 15 are each connected between the top electrodes of a neighboring cMUT cell except in the case of cells on the periphery. Thus the centrally positioned cell 2 connects to six other cells while cells on the periphery connect to three other cells. The bottom electrodes 10 of each cell 2 are also electrically coupled together and are connected with bottom electrodes of other acoustical subelements 16 to form a common ground plane. As a result, each acoustical subelement 16 is wired to operate as an acoustic transducer that is seven-times-larger than one hexagonal transducer cell 2. Individual cells 2 within an acoustical subelement 16 cannot be reconfigured to form different subelements. Other embodiments of the invention are not so limited.

In general, reference to an “acoustical subelement” corresponds to the smallest independently controlled acoustical unit. This may mean multiple ones of the cells 2, which are electrically connected to one another such that they cannot be reconfigured, as shown in FIG. 1, or can mean a single cell 2 if the cell is an independently controlled acoustical unit.

A reason for electrically grouping the transducer cells 2 into acoustical subelements 16 is that it is difficult to provide electronics that allows individual control over the relatively small individual transducer cells. That is, while the acoustical performance with the relatively small membrane size corresponding to an individual cell 2 is both excellent and desirable, operation and control of the transducer cells is effectively had at a higher level, e.g., that of the subelement comprising multiple individual transducer cells. For further explanation see U.S. Pat. No. 6,865,140 which is now incorporated herein by reference. However, features of the invention can nonetheless be realized in embodiments which depart from such arrangements.

Although illustrated as hexagonal, the individual cells 2 can have circular, rectangular or other shapes about the peripheries. Hexagonal shapes effectively provide dense and uniform packing of the cMUT cells 2 to form clusters of the transducer subelements 16. In still other embodiments, the cMUT cells 2 in each acoustical subelement may have different dimensions so that the acoustical subelements will have composite characteristics of the different cell sizes, giving the transducer a broadband characteristic.

The term “subelement” (without the qualifier “acoustical”), as used herein, means the combination of an acoustical subelement and its associated integrated electronics, e.g., formed in an adjoining ASIC. An “element” is formed by connecting acoustic subelements together using a switching network. The elements can be reconfigured by changing the state of the switching network. The switching network may be configured by programming a first series of switches that connect a limited number of subelements for collective operation in groups of subelements. The subelements within a group are controlled with the same timing signals for generating high voltage pulses to drive associated transducer cells 2. A second series of switches, e.g., conventionally referred to as a cross point array, can define an overall shape of the element by wiring together the groups of subelements. At least some of the switches included in the switching network are part of the associated integrated electronics. According to several embodiments of the invention, all of the switches are part of the associated integrated electronics, being formed on one or more ASICs which provide circuit support functions for the array of transducer cells.

Ring-like shapes and other element shapes, suitable for synthesizing acoustic waves, can be configured with the programmable switching network. The subelements can be reconfigured by changing the state of the switching network to interconnect different subelements to one another. According to the disclosed embodiments, it is not necessary to connect every subelement directly to a switchable channel line. Rather, it has been found convenient to apply switching circuitry to electrically connect a limited number of subelements into groups and to connect the support circuitry of one associated subelement in each group to a switchable channel line. With such an arrangement appropriate timing signals are fed to all of the subelements in a group via the connection of the one subelement to the channel line. In this way, given a fixed number, N, of timing pulse channel lines, the switching and control circuitry can be deployed to connect each subelement to any one of the channel lines without having to route all of the N channel lines for direct connection to each subelement. That is, given an array of subelements arranged in rows and columns, and a total of N channel lines, e.g., N=32, a limited number of the channel lines, e.g., four, can be arbitrarily selected via switches to route their signals along each row of subelements for selective connection to each subelement in the row. This scheme, in combination with programmable switches that electrically connect adjoining subelements into groups, can provide necessary flexibility to connect various groups of the subelements to any of the N channel lines.

Reconfigurabilty of acoustical subelements in a two-dimensional array to form elements, such as annular rings, is described in U.S. Pat. No. 6,865,140 ('140) incorporated herein by reference. One form of such reconfigurability is referred to as the mosaic annular array, which involves building annular elements by grouping acoustical subelements. As described in the '140 patent, reconfigurability can be effected with a digital scanning architecture for configuring connections with switches. According to the connections, the pulser timing signals are transmitted among the transducer subelements to provide multiple operating states that each generate a different wave pattern. This programmable switching network is used to sequentially generate a series of wave patterns across the two-dimensional array of acoustical subelements in order to form a scan or an image based on a sequence of reflections therefrom. Most elements that can be defined with the switching network are contiguous groups of interconnected subelements. For a given element geometry, the switching network is programmable to connect a set of acoustical subelements to receive timing pulses from a particular system channel. Portions of the switching network may be placed directly in the substrate upon which the cMUT cells are constructed. That is, since cMUT arrays are built directly on top of a silicon substrate, the switching electronics can be incorporated into that substrate. According to the invention, the switching network can also be formed in an ASIC comprising the subelement support circuitry.

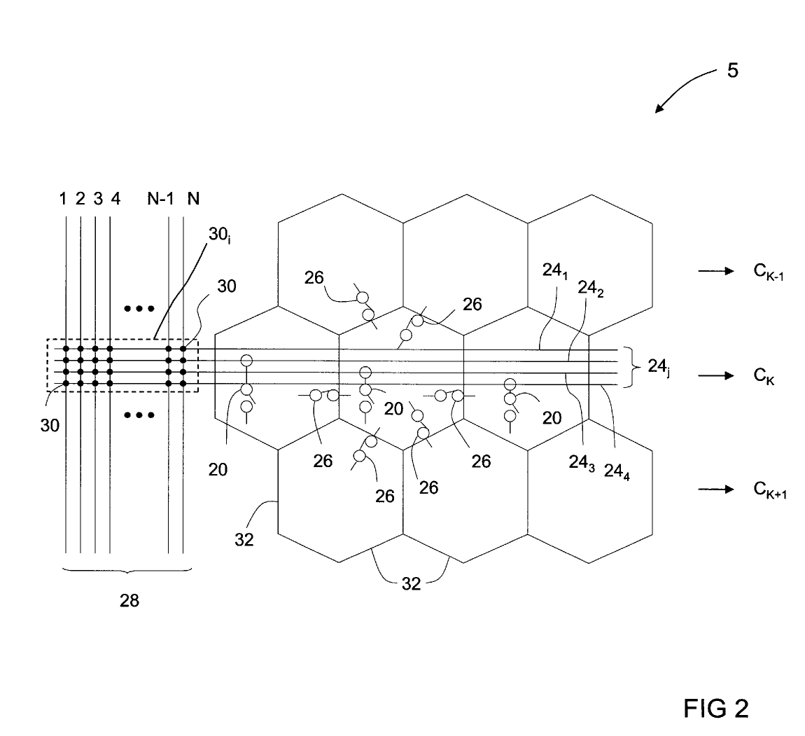

One implementation of a reconfigurable cMUT array 5 is schematically shown in the embodiment of FIG. 2 wherein, generally, a series of N system row channel lines 28 are each connectable to individual ones in a set of column access lines to effect selectable connections between individual channel lines and subelements. For simplicity of presentation, various switches are shown with respective selectable connections, while the corresponding control lines that transition the switches between open and closed modes are not illustrated, these being well-known arrangements.

Column access switches 20 are each positioned to connect a given acoustical subelement 32 to one of several column access lines, e.g., column lines 24_{J} (J=1, 4). The column access lines 24_{J} are connected to the system channels using a cross-point switching array comprising a plurality of timing channel select switches 30_{I} (I=1, JN) in each row. Although the timing channel select switches 30_{I} are shown in a matrix form, the matrix representation is only conceptual and, according to embodiments of the invention the channel select switches 30_{I} may not have a physical layout corresponding to conventional rows and columns in a matrix. This connection arrangement is directly applicable to the mosaic annular array described in the '140 patent. In such a device multiple rings can be formed, with each ring element connected to a single system channel. The desired configuration can be effected by programming of the access switches 20, each of which is connected to a column access line, which is in turn connected through a timing channel select switch 30_{I} to one of the N system channel lines 28. In the embodiment of FIG. 2, the access switches are arranged in a serial sequence wherein each subelement contains only one access switch 20 and wherein every four consecutive subelements in each row are connectable through a switch 20 to a different one of the four column access lines 24_{J}. This arrangement reduces the number of access switches required for a given number of column access lines 24_{J}. The required number of timing channel select switches 30_{I} and column access lines, J, is determined in part by the array size and the application.

For an example embodiment FIG. 2 schematically illustrates pertinent features of subelements and associated control wiring that effect formation of a ring element. Multiple acoustical subelements 32 in a two dimensional array 5 are positioned in Columns C. The subelements 32 may be in accord with the design of subelement 16 shown in FIG. 1 or may assume other embodiments as already described. For purposes of continuity of description, like reference numbers for features shown in FIG. 1 correspond to like features in FIG. 2. Details for one exemplary column C_{K} are shown in FIG. 2, wherein a single access switch 20 is allocated for each acoustical subelement 32 and four column access lines 24_{J} pass along each column C of acoustical subelements 32 in the array. Each column has a unique address and multiple elements may be simultaneously defined.

Matrix switches 26 are positioned to selectively connect electrodes 12 (shown in FIG. 1) of different subelements to one another via the conductors 15. With three exemplary matrix switches 26 positioned in each subelement, the associated acoustical subelement 32 is electrically connected to an access switch 20 for that subelement and to the three matrix switches 26 associated with that subelement and to three matrix switches associated with three neighboring subelements. With this arrangement and an appropriate switching configuration, a signal that travels through timing channel select switches 30_{I} to a first subelement and then through a matrix switch 26 to an adjoining subelement enables the acoustical subelement 32 associated with the second subelement to be connected to a system channel through the first subelement. This also means that an acoustical subelement 32 may be connected to a system channel even though it is not directly connected via an access switch 20. It is to be understood that FIG. 2 is a schematic illustration and is not specific to any particular embodiment or layout of the electronics associated with the subelements. In the past some of the switches, e.g., switches 20 and 26 have been placed in the subelements, e.g., within individual circuit cells of an ASIC that each contain the support circuitry of a subelement. In contrast to this, the timing channel select switches 30, have been placed outside of the array 5, i.e., in an electronics unit external to the probe unit which houses the array 5 or in portions of the circuit paths between the electronics unit and the ASIC array containing the subelement support circuitry.

While FIG. 2 shows three matrix switches 26 per subelement, it is also possible to have fewer than three matrix switches to conserve area or to allow room for larger switches which have lower on resistance and which consume more area. There may also be more than three matrix switches per subelement in which case the peripheral shapes and sizes of the transducer cells may be varied or different than that of the uniform hexagonal cells 2 of FIG. 1. A feature of the architecture shown in FIG. 2 is that matrix switches can be used to route around a known bad subelement for a given array. In the past, with an array of acoustical subelements having a hexagonal shape, as shown in FIG. 2, the corresponding subelements formed on an adjacent ASIC have been formed with the same shape, it being understood that columnar or rectangular shapes for acoustic subelements 32 are also possible and these might require fewer switches.

FIG. 2 describes a functional circuit arrangement according to which a switching network might work for a particular prior art architecture such as described in U.S. Ser. No. 11/172,599 (filed Jun. 29, 2005, and incorporated herein by reference), as well as for a layout architecture in accord with the present invention, wherein the column access lines 24_{J} (J=4), extend along the column C_{K} of acoustical subelements 32. While only three acoustic subelements 32 are shown in this column, it should be understood that many other subelements extend along this column. The column access lines 24_{J} are alternately connectable to each one of the N system channel lines 28 by means of the timing channel select switches 30_{I} (I=1, JN), which provide a cross-point switching matrix function. FIG. 2 is to be understood as presenting a schematic illustration of functional relationships and is not limited to any particular physical layout. In corresponding physical layouts according to the invention: the column lines 24_{J} do not extend beyond the end of a row R in order to connect with the select switches 30_{I}; and the timing channel select switches 30, are not physically positioned outside the array of ASIC cells.

As seen in FIG. 2, each column access line 24_{J} can be connected to any one of the system channel lines 28 by closing an appropriate one of the select switches 30_{I} and opening the select switches 30_{I} that could otherwise connect the particular access line to the other system channel lines 28. In the past the circuit electronics corresponding to the functions performed by the select switches 30_{I} has been outside of the ASIC area consumed by the subelement support circuitry so as to not be restricted in size. Due in part to the area required for this function, the cross matrix switch function performed by the channel line select switches 30_{I} has not been placed within the ASIC cell areas allocated for subelement support circuitry.

Although FIG. 2 shows a fully populated cross-point switching matrix, in cases wherein it is not necessary to have switches that allow every access line 24J to be connected to every channel line select switch 30_{I}, a sparse cross-point switching matrix can be used in which only a small subset of the system channel lines 30 can be connected to a given column access line 24_{J}, in which case only some of the timing channel select switches 30_{I} depicted in FIG. 2 would be present.

The access switches 20 are so named because they each give a subelement direct access to a column line 24_{J}. In the exemplary implementation depicted in FIG. 2, there are six other switch connections for each subelement, these taking the form of the aforedescribed matrix switches 26 which each allow an acoustical subelement 32 to be connected to a neighboring acoustical subelement 32. For embodiments wherein each subelement is formed with support circuitry formed in a separate ASIC cell, and in which there are six connections between each acoustical subelement and the adjoining subelements (e.g., for the hexagonal pattern of FIG. 1), only three switches reside in each ASIC cell of a subelement while the other three switches 26 are positioned in the ASIC cells of neighboring subelements. Thus there may be a total of four switches with associated digital addressing and control logic (not shown) positioned in the ASIC support circuitry of each subelement. This is just one exemplary implementation. The number of row lines 24_{J}, the number of access switches 20, and the number and topology of the matrix switches 26 could vary without departing from the general concept disclosed in Ser. No. 11/172,599. Although the access and matrix switches can be fully integrated with other acoustical subelement support circuitry on an ASIC, these switches may be fabricated within the same semiconductor substrate on which the MUT array is to be fabricated.

U.S. patent application Ser. No. 10/697,518, entitled “Methods and Apparatus for Transducer Probe”, discloses an embodiment wherein high-voltage pulsers may be integrated into the handle of an ultrasound transducer probe and then activating those pulsers using timing signals that pass through a low-voltage switching matrix positioned outside the ASIC subelement array, also incorporated in the probe. The present invention provides an alternate architecture that implements the switching matrix function described with respect to FIG. 2 with timing channel select switches 30, in combination with the access switches 20 and matrix switches 26.

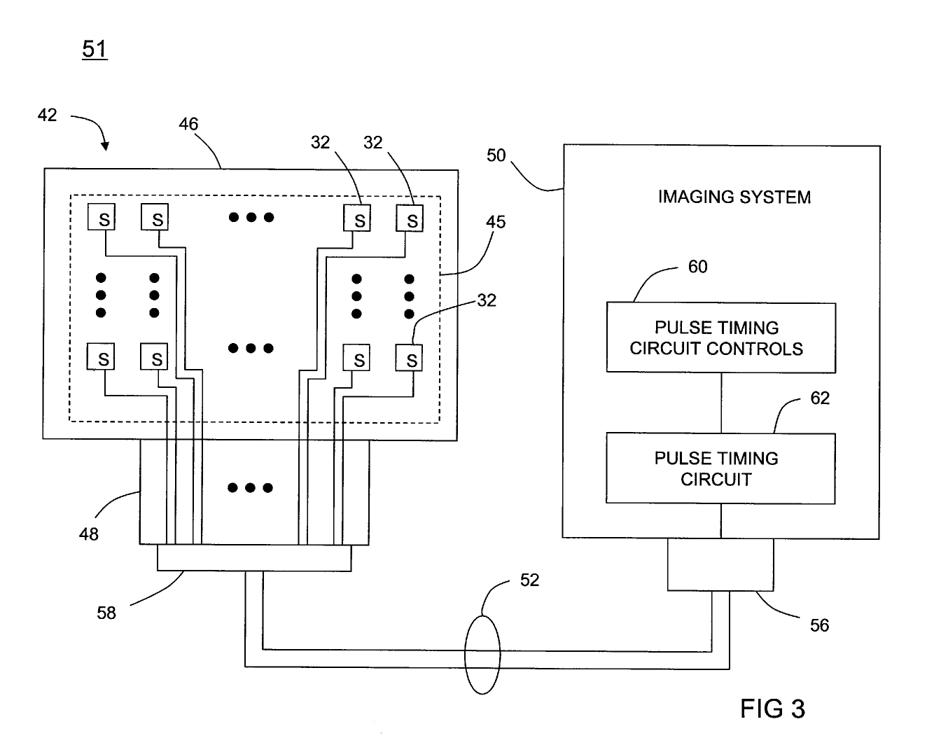

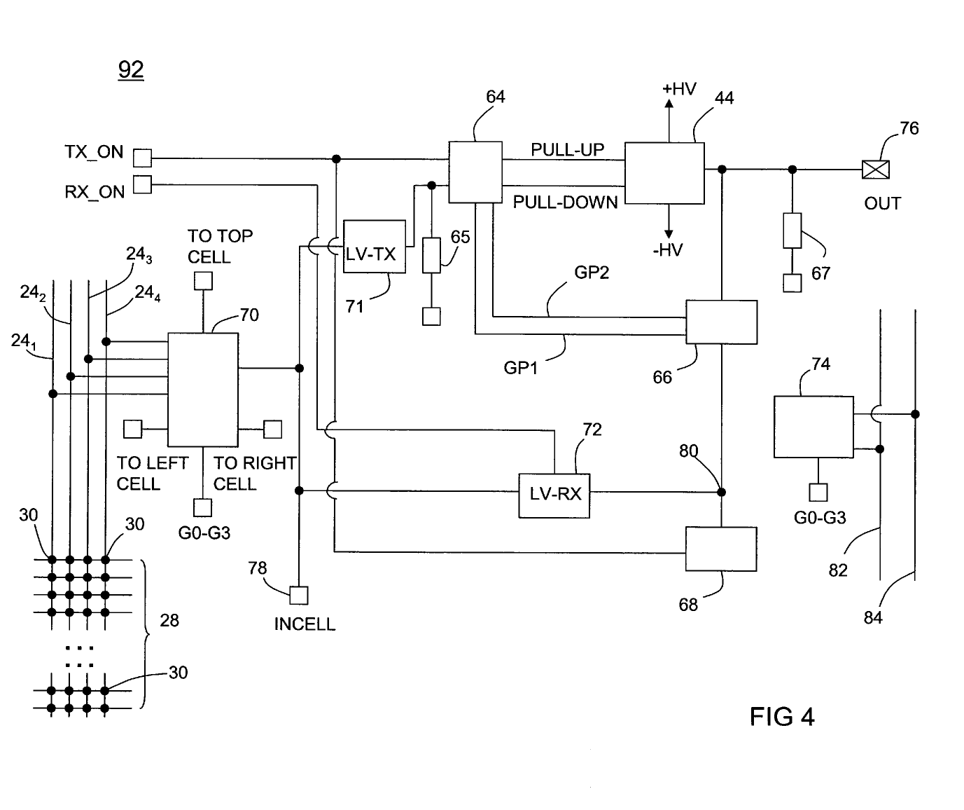

FIG. 3 illustrates a probe unit 42 in combination with a remote electronics unit 50 which together form a complete ultrasound system 51. The probe unit includes a probe head 46 for housing an array 45 of subelements S formed as a combination of ASIC circuitry and acoustical subelements 32 wherein the timing channel select switches 30, are integrated with the ASIC circuitry. The probe unit includes a probe handle 48 that may contain wiring or circuitry which is not integrated into the subelement ASIC circuitry. The probe unit and the electronics unit are connected to one another via one or more cables 52 each comprising a multiplicity of electrical conductive leads for transmitting a variety of I/O signals between the probe unit 42 and the electronics unit 50. The I/O signals carried on the cable 52 include power, timing and control signals and imaging data. Each cable 52 is coupled to the imaging system 50 and to the probe 42 by respective cable connectors 56 and 58. The electronics unit 50 houses imaging system electronics which includes pulse timing circuit controls 60 and pulse timing circuits 62 that provide timing signals along each of the channel lines 28 (see FIG. 2) to effect selective connections with combinations of subelements S to create elements or apertures. Conventional components and circuitry in the electronics unit 50 for processing the data to generate an image are not shown in the figure. Other embodiments according to the invention may place the pulse timing circuit controls 60 or the pulse timing circuits 62 in the probe unit, e.g., in the probe handle 48. Support circuitry associated with each subelement S is formed in an ASIC cell that includes the aforedescribed access switches 20 and matrix switches 26 as well as other circuitry described herein with reference to FIG. 4. Further, some of the ASIC cells may include one or more of the timing channel select switches 30_{I}. According to one series of embodiments, with the support circuitry of each subelement S formed in a separate ASIC cell, individual ones of the timing channel select switches 30_{I} are formed within individual ones of the ASIC cells.

When the system 51 is in the transmit mode, each subelement S is programmably configured to define part of an element. The subelements receive a timing pulse signal through a channel line 28 that is coupled thereto through a channel select switch 30_{I}. Pulser circuits in each subelement are triggered by the timing pulse to generate the high voltage signal required to radiate ultrasound energy from the associated acoustical subelement 32. Parameters of each respective pulse train sent in each channel line are varied to achieve focused ultrasound beam transmission via acoustic radiation from multiple ones of the elements. The pulse timing circuit 62 generates multiple low-voltage transmit control (i.e., timing) signals that are carried by the coaxial cable 52 from the imaging system in the electronics unit 50 to the probe 42. Once the low-voltage transmit control signals reach an individual subelement S, they are decoded and used to control the local high-voltage pulser circuits to drive individual acoustical subelements 32 in the transmission mode. The pulser circuits cause the acoustical subelements to generate radiation according to the transmit control signals. The pulsers circuits 44 may be unipolar, bipolar, or multi-level pulsers, or a combination thereof. Placing the pulsers in the ASIC circuitry of the subelements S advantageously permits pulse timing circuitry 62, controlled by the pulse timing control circuitry 60, to be located either in the imaging system 20, as shown in FIG. 3, or in the probe handle (not shown).

With appropriate ones of the low voltage switches 20, 26 and 30_{I} closed, the timing signals reach the pulser circuits to trigger the generation of the ultrasound signals. The low-voltage switches 20, 26 and 30_{I} are reprogrammable between consecutive transmissions to receive echo signals and generate new patterns. Routing of the timing signals is such that all subelements S that are part of a given transmit element are electrically connected together (i.e., in common) to receive the same low-voltage transmit control signal. Similarly all subelements S that are programmed via appropriate switches to be part of a given receive element are electrically connected together such that their receive signals contribute to the net receive signal for that element.

In accordance with one series of embodiments of the present invention, the ultrasound system 51 comprises a multiplicity of acoustical subelements 32, and a multiplicity of electronic cells, e.g., ASIC cells, incorporated in the probe unit 42. Each electronic cell contains support circuitry to interface a respective acoustical subelement 32 to imaging system electronics in the unit 50 to which the probe is connected via the cable 52. An electronics cell that facilitates the sending of pulses to a respective acoustical subelement 32 during transmission and the receiving of echo signals from the respective acoustical subelement 32 during reception is referred to herein as an ASIC cell 92.

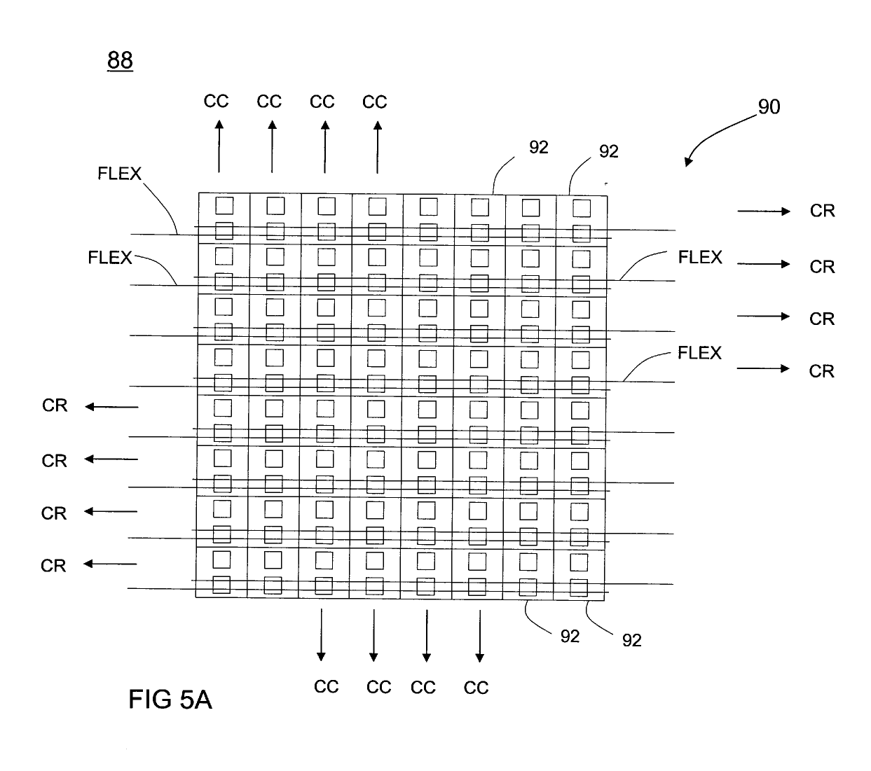

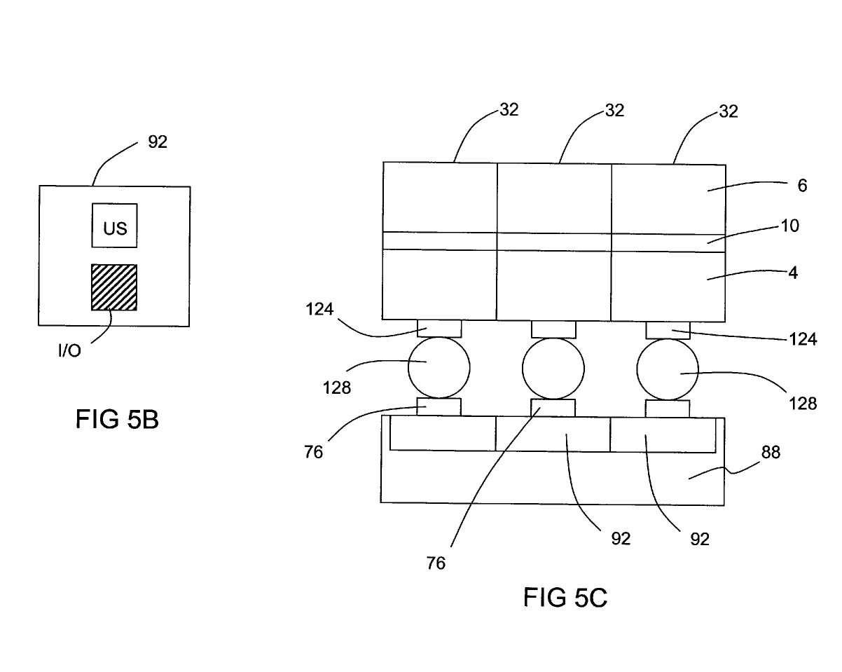

FIG. 4 is a simplified block level diagram showing a partial representation of circuitry in an individual ASIC cell 92 in accordance with embodiments of the invention wherein one ASIC cell 92 provides support circuitry to one acoustical subelement 32. Each ASIC cell 92 comprises a high-voltage pulser circuit 44, a low-voltage switching network 70, a low-voltage splitter 64, a high-voltage Transmit/Receive (T/R) switch 66, a low-voltage ground switch 68, a low-voltage transmit switch 71, a low-voltage receive switch 72, and a low-voltage digital control circuit 74. The low-voltage receive switch 72 (labeled LV_RX in FIG. 5) is situated between the center node 78 and the T/R switch 66. The low-voltage transmit switch 71 (labeled LV_TX in FIG. 5) is situated between the center node 78 and the splitter 64.

The ASIC cell 92 is placed in a transmit mode upon receipt of a global transmit mode control signal at node TX_ON from the imaging system. This global transmit mode control signal TX_ON is received by splitter 64, ground switch 68, and low-voltage transmit switch 71. RX_ON is the inverse of TX_ON and is used to control the low-voltage receive switch.

The low-voltage switch network 70 corresponds to one access switch 20 and three matrix switches 26, as previously described with reference to FIG. 2. These switches do not have to withstand high voltage and so can be much smaller than equivalent-on-resistance high-voltage switches. In the transmit mode, pulser timing and control signals from the pulse timing circuit 62 (see FIG. 3) are sent to the low-voltage switch network 70. The pulse timing and control signals may be sent directly to the ASIC cell 92 via a column access line 24_{J} and the access switch 20 incorporated in the switching network 70. Alternately, the pulse timing and control signals may be sent to an ASIC cell 92 via the column access line 24_{J} of a different ASIC cell 92 and through a matrix switch 26 incorporated in switching network 70.

In the transmit mode, the splitter 64 receives the pulser control signals from the system (via the switching network 70 and the low-voltage transmit switch 71) in the form of low-voltage bipolar pulses. A circuit element 65, which may be a resistor or a transmission gate connected to ground, maintains the input of the splitter at a known value, e.g., ground. This function could also be performed using an analog switch to ground that is controlled by RX_ON. Use of such a low-voltage switch would greatly reduce the amount of area for this function. The splitter 64 splits each bipolar input pulse sequence into two pull-up and pull-down pulse sequences that control the high-voltage pulser circuit 44.

The high-voltage pulser circuit 44 is located between the splitter 64 and a high voltage output node (i.e., signal pad) 76 also illustrated in FIG. 4. The output node 76 is in turn connected (not shown) to drive the associated acoustical subelement 32. The pulser circuit drives the output node 76 and its load between +25 V and −25 V as a function of the pull-up and pull-down pulse sequences issued from the splitter 64. A circuit element 67, which may be a resistor or a transmission gate connected to ground, biases the acoustical subelement 32.

In addition, two other outputs (GP1, GP2) of the splitter 64 carry control signals for the high-voltage T/R switch 66. The high-voltage T/R switch 66 is located between the output node 76 and the low-voltage receive switch 72, as shown in FIG. 4. The purpose of T/R switch 66 is to protect the low-voltage circuitry from the pulser output, which is a high voltage during the transmit cycle. During transmit, the T/R switch 66 is turned off to isolate the low-voltage circuitry from the high transmit voltage.

The ground switch 68 is a low-voltage analog transmission gate that is used between the high-voltage T/R switch 66 and ground. During transmit, the ground switch 68 is closed in order to hold the receive channel at a preset level and the low-voltage receive switch 72 is open. The high impedance configuration of the low-voltage receive switch 72 prevents the ground switch 68 from affecting the low-voltage transmit control signal. Whenever the low-voltage transmit control sequence returns to zero, the splitter 64 outputs control signals GP1 and GP2 to cause the T/R switch 66 to close. This action allows the output node 76 to discharge either from a high voltage state or a low voltage state to the ground level.

Once the transmit cycle is finished, the global control signal TX_ON changes level, causing all of the ground switches to be turned off, which allows the receive channels in all of the ASIC cells 92 to float in preparation for the receive cycle. In the receive cycle, all of the high-voltage T/R switches in the array are closed. In addition, the low-voltage transmit switch 71 is open and the low-voltage receive switch 72 is closed. This configuration allows the low-voltage receive signals from the transducers to be routed to the low-voltage switching network 70, but prevents them from affecting the splitter. The low-voltage switching network 70 routes the signals back to the electronics unit 50 over the same channel lines 28 that were previously used to drive the low-voltage transmit control signals. Once the receive cycle is completed, the array is reconfigured for the next transmit cycle.

Exemplary circuitry for the components shown in FIG. 4 are provided in Ser. No. 11/172,599. See, also, U.S. patent application Ser. No. 10/751,290 entitled “Alignment Method for Fabrication of Integrated Ultrasound Transducer Array” also incorporated herein by reference, which describes design of CMOS circuit cells to match the geometry, e.g., hexagonal shapes, of the array 5 of acoustical subelements. See, also, U.S. Pat. No. 6,759,888 which describes further details of operation of circuits shown in FIG. 4.

In the transmit mode the digital control circuitry 74 sends program data in the form of signals that control the state of the access switch 20 and matrix switches 26 of the associated low-voltage switching network 70, forming the transmit aperture by connecting subelements. Control of which pulsers are fired is set entirely by the dense low-voltage switching matrix. Configuration or reconfiguration of the switches also dictates how signals are routed in the receive state. Both of these switch states (transmit mode and receive mode) are stored locally in the digital control electronics, e.g., in SRAM. Additionally these settings may be revised from transmit mode to transmit mode as well as from transmit mode to receive mode. Further, the settings may be altered during the receive cycle in order to allow for multiple focal zones during the receive state.

Both the digital and analog signal lines may be brought into the ASIC cell 92 along a single face of the ASIC, extending in a direction parallel with a plane along the transducer array 5 and columns of cells. FIG. 4 illustrates the cell matrix addressing architecture with one selected low-voltage column line 24_{J}. Digital switch state data is programmed into the array on multiple four-bit data bus lines 82. Each column of subelements S has a unique address and rows are programmed in parallel. Each subelement S in each column is connectable to any of the four column lines 24_{J} through a combination of access switches and matrix switches. Each column of subelements S has one digital address select line 84 that selects all cells on that column in parallel simultaneously. Once the cells are selected, they all latch the data on their respective column bus lines 82 simultaneously. The digital control stores the data transmitted on the data bus 82. Typically only one face of the array can be used for pad access. In the past, the digital select lines 84 were brought in via metallization formed on the associated ASICs along the subelement rows and then converted to run across the columns. This can be done using junctions that tie the column address select lines 84 to the row address select lines. There may be two address lines in each column and two junctions to respective column lines in each row. See, again, Ser. No. 11/172,599 with reference to FIGS. 20-22 therein. The use of address select lines integrated in each column of the array simplifies the digital circuitry in each cell since there is no need for an address decoder and no need for a latch to store the addressed state. The select line and other addressing methods are disclosed in U.S. patent application Ser. No. 10/978,012 filed on Oct. 29, 2004 “Method and Apparatus for Controlling Scanning of Mosaic Sensor Array” hereby incorporated by reference. Other addressing methods disclosed in that filing could also be used with the present invention.

The aforedescribed illustrations have described support circuitry for acoustical subelements without reference to specific ASIC architectures for supporting the transmit and receive functions for acoustical subelements 32. For example, in FIGS. 2 and 4 numerous components such as access switches 20, matrix switches 26 and control logic are functionally described to provide an understanding of the roles of various circuit components supporting each acoustical subelement 32, but without reference to the layout of individual components in a circuit structure.

The ASIC cells in the array 46 of subelements S may be integrally formed in the substrate upon which the cMUT cells are constructed, but as illustrated herein can also be in a different substrate adjacent the substrate on which the acoustical subelements 32 are formed. Specifically, the ASIC cells may be fabricated on a plurality of adjoining ASICs 88 which are positioned along a plane in which the acoustical subelements 32 are positioned. FIG. 5A is a schematic view of a portion of an array 90 of ASIC cells 92 according to one embodiment of the invention. In this partial plan view of an ASIC 88, a small block of the cells 92, shown arranged in eight cell rows CR and eight cell columns CC, is an exemplary portion of a much larger block of ASIC cells 92 as suggested by arrows extending from rows CR and columns CC.

In the ultrasound imaging system 51 there may be multiple ASICs 88 formed in ASIC rows and ASIC columns with each ASIC 88 providing an array 90 of the ASIC cells 92. Each ASIC array 90 may comprise a very large number of ASIC cells 92 arranged in perhaps hundreds or an even larger number of such cell rows CR and cell columns CC. Each ASIC cell 92 provides circuit support functions, such as illustrated in FIG. 4, for at least one acoustical subelement 32, with the combination of the cell 92 and associated acoustical subelement 32 forming a subelement S. The illustration of FIG. 5A is presented in a schematic form such that individual features most relevant to the invention, such as metal lines and contact pads, are shown in a single view even though they may reside in different planes along the ASIC 88. Generally the ASIC cells 92 are shown as uniform rectangular-shaped units although they may be hexagonal or of another shape. Referring also to FIG. 5B, in a simplified and enlarged plan view, one such ASIC cell 92 is shown to include at least a first contact pad US and an optional second contact pad I/O. The contact pads US and I/O are positioned in metallization above circuitry in the cell 92. By way of example, the pads may be rectangular shaped and dimensioned on the order of 50 microns on each side, while the rectangular cell 92 may be on the order of 150 microns on a side. The pads, formed in an uppermost level of ASIC metallization may connect directly with the underlying cell and/or may extend through metallization to connect with circuitry in other cells 92. Generally, the geometry allows placement of the contact pads over the circuitry in the ASIC cells 92 to route signals into or out of one or more of the cells 92.

The contact pads US effect connection between the circuitry in each cell 92 and the associated at least one acoustical subelement 32 to (i) carry a high voltage signal from a pulser circuit 44 to the acoustical subelement 32 during the transmit mode, and (ii) to carry an echo signal from the acoustical subelement 32 to the ASIC cell 92 during the receive mode.

FIG. 5C is a simplified partial cross sectional view of the subelement array 46 illustrating three acoustical subelements 32 formed on a substrate 4, and three associated ASIC cells 92 along a portion of an ASIC 88. Each acoustical subelement 32 is connected to an ASIC cell 92 via electrically conductive bumps 128 and electrically conductive pads 76 and 124. Other interconnect techniques such as anisotropic conductive paste (ACP), anisotropic conductive film (ACF), electrically conductive polymers, metallized bumps, vertical interconnect systems, e.g., z-axis interposers, flexible printed circuits, etc. or metallized vias could also be used. The cells 92 may each include a second contact pad I/O which provides input or output signals between the ASIC cells and circuitry external to the ASIC 88, e.g., in the probe handle 48 or in the remote electronics unit 50.

The contact pads I/O provide a level of versatility and integration to reduce the size and weight of electronics in the probe unit 42. In prior designs of ultrasound probes having support circuitry provided on ASICs in the probe units, the ASICs have been partitioned into a cell block region containing an array of ASIC cells providing support circuitry dedicated to operation of acoustical subelements, and one or more I/O block regions allocated for transmission of signals and placement of protection circuitry. The I/O signals include power, timing and control and image data which are transmitted between support circuitry in the ASIC cells and electronics external to the probe unit, e.g., in the electronics unit 50. Typically, such allocated I/O regions have been positioned along the periphery of the ASICs. In such arrangements signals are communicated between the ASIC cells and the I/O regions through metallization levels in the ASIC. However, the area along peripheral ASICs which is consumed for these functions can disrupt continuous alignment between individual cells of the ASIC support circuitry and the transducer cells. In order to avoid such disruption in pitch among transducer cells, the circuitry may include one or more layers which perform redistribution functions to accommodate differences in pitch between the ASIC cells and associated transducer acoustic subelements. See U.S. Ser. No. 11/743,391, filed May 2, 2007, now incorporated herein by reference. In lieu of providing a separate region for I/O transmission on an ASIC that provides the support circuitry for the acoustical subelements, one embodiment of the invention integrates the I/O circuitry and I/O connections into the ASIC cells 92.

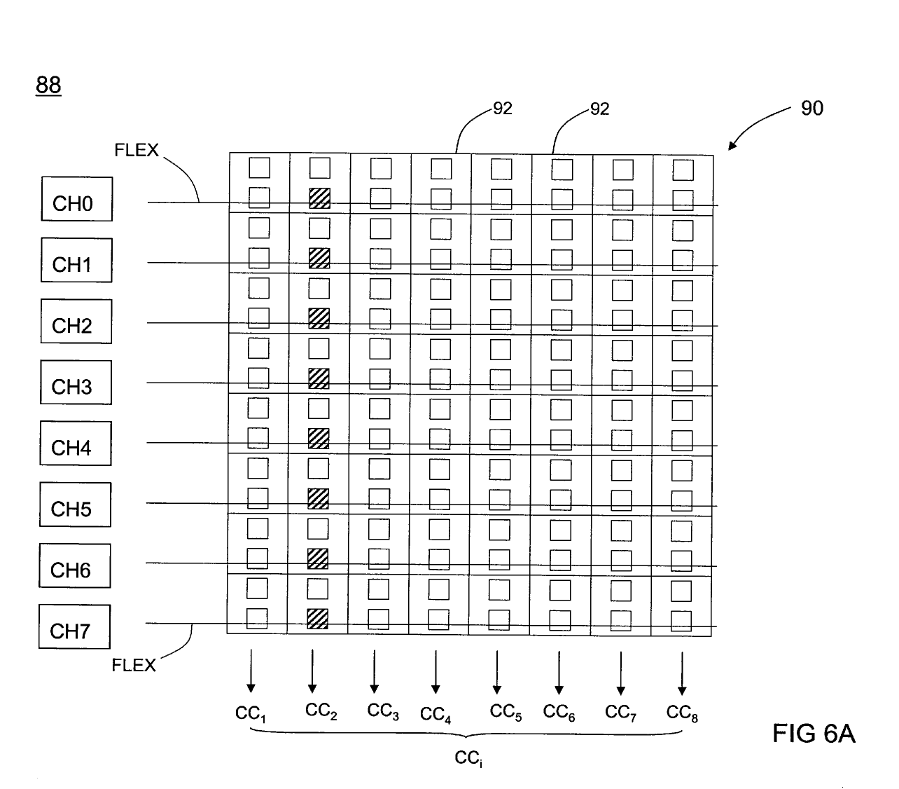

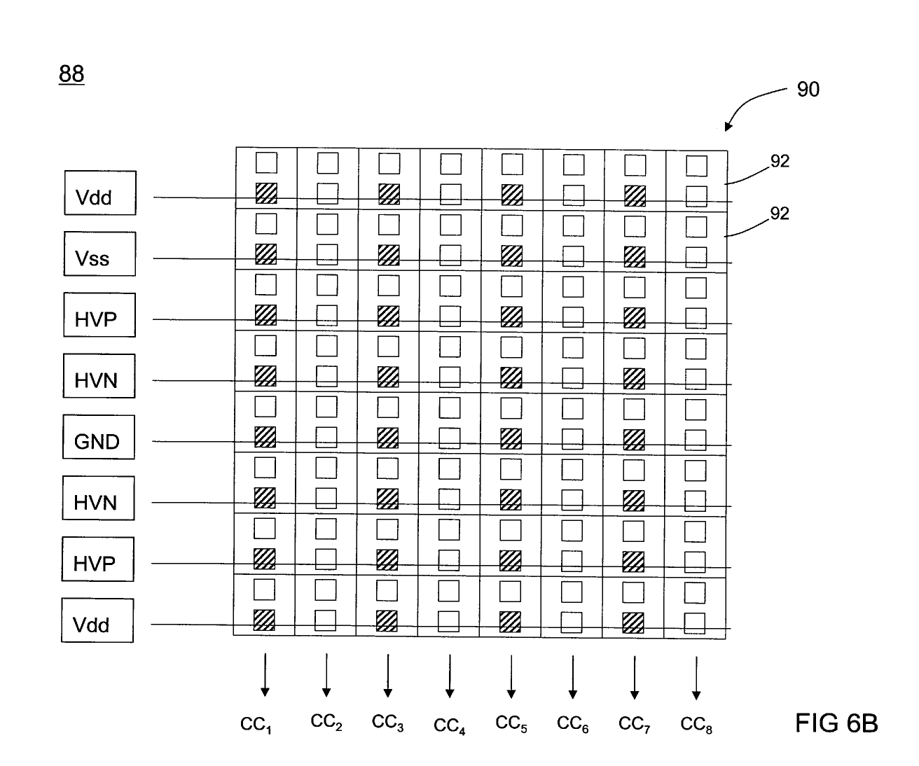

FIGS. 6A and 6B illustrate exemplary arrangements for routing of several types of I/O lines into the array 90 of ASIC cells 92 in the ASIC 88. For reference purposes each of the ASIC cell columns CC is consecutively numbered from 1 to 8 from the left side of the array 46. Placement of a contact between an I/O line and a cell 92 to bring a signal into or out of the cell is indicated by cross hatching of the I/O contact pad.

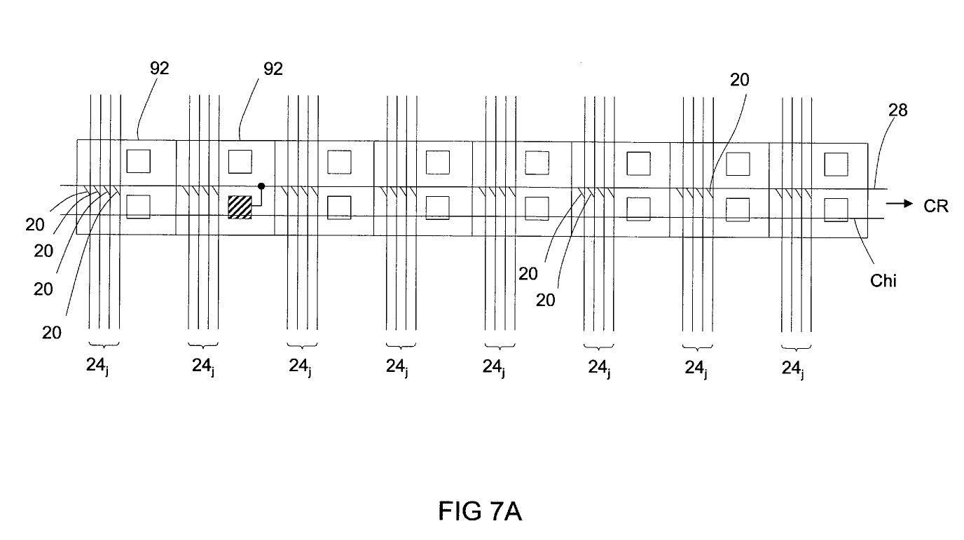

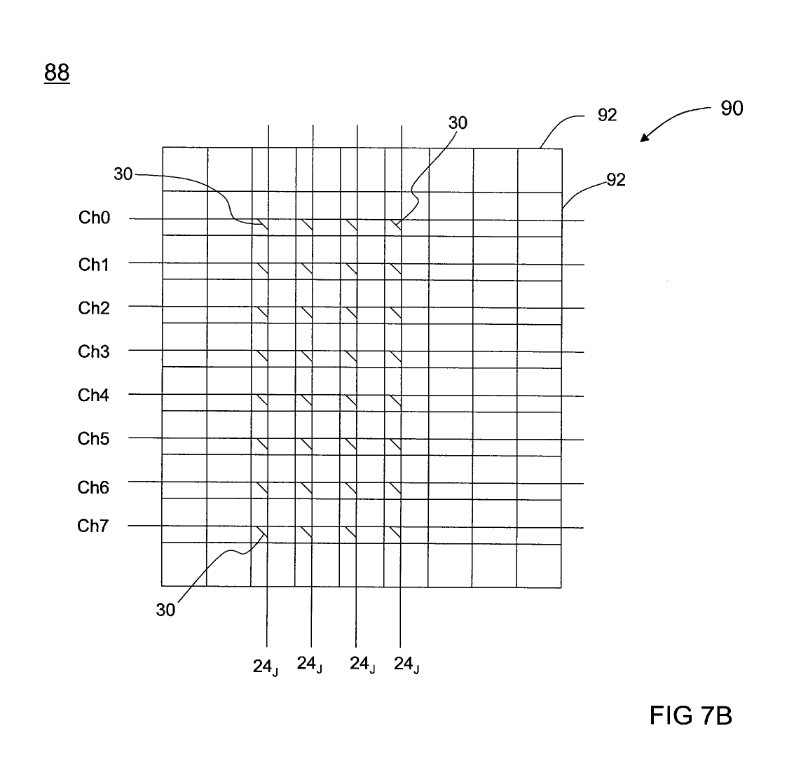

In FIG. 6A each of eight channel lines 28 (Ch0-Ch7) is brought from a flex circuit line, adjacent the metallization layer of the ASIC 88, through a contact pad I/O and into one of the cells 92 in a second column CC2 of the array 90. According to this embodiment a timing channel select switch 30, for selectively connecting a channel line 28 to one of the column access lines 24_{J} (see FIG. 7A) is formed in another one of the cells 92 in a different column CCi. In this manner selective connection between each channel line 28 and one of the column access lines is effected. The access lines 24_{J} may be brought into the ASIC cell 92 containing the timing channel select switches 30_{I} through routing in the ASIC metallization. Thus there is no longer a need to extend the access lines 24_{J} outside of the ASIC array 90, e.g., off chip. Rather, as shown for an exemplary column CC of ASIC cells 92 in FIG. 7A, one or multiple, e.g., four, access line switches 20 may be placed in each cell 92 to effect connection of the cell 92 to one of the four column access lines 24_{J}. Thus for an exemplary eight channel lines 28 and four column access lines along each column CC, a total of 32 timing channel select switches 30_{I} (I=1, JN=32) can be formed within the ASIC cells 92. The channel select switches 30, may be placed in multiple ones, e.g., up to 32, of the ASIC cells 92. See FIG. 7B which illustrates another exemplary routing of channel lines 28 into a 4×8 block of cells 92 which each contain one of the channel select switches 30_{I}. In these embodiments the channel lines may each be initially brought to the ASIC array 88 via flex circuit lines (e.g., from a pulse timing circuit 62 located in the probe handle 48 or in the electronics unit) to one of the contact pads I/O per FIG. 7A, and then routed to other cells 92 such as shown in FIG. 7A in which the channel select switches 30, are formed. Thus the channel lines 28 may be brought into ASIC cells 92 located along one column or a combination of cell columns CC and routed to other cells 92 in which the channel select switches 30, are positioned for connection with column access lines 24_{J}.

Multiple ones of the ASICs 88 may be formed in rows and columns to form a larger array 90 of ASIC cells 92 wherein rows in adjoining ASICs are aligned to form larger rows extending among the ASICs and columns in adjoining ASICs are aligned to form larger columns extending among the ASICs. The channel lines 28 can extend along the larger rows from ASIC to ASIC with flex circuitry.

In FIG. 6B numerous power lines are shown extending from outside the array, brought through contact pads I/O in columns 1, 3, 5 and 7, into each of the underlying cells 92. In addition to Vdd, Vss and ground (GND), the power lines include high voltage for P-type power devices (HVP) and for N-type power devices (HVN). Based on design considerations, the repetition frequency of I/O contacts between and along the array columns will vary among the different types of individual power lines, and the illustrated distribution is exemplary and not limiting. Numerous other I/O lines may also be integrated into the array 90 based on flex connections to contact pads I/O positioned above the ASIC cells 92. Other examples include data lines (analog or digital), control lines and select lines. Depending on feature sizes and design requirements, it may be desirable to place more than two contact pads above each ASIC cell 92. In other instances it will not be necessary to have multiple contact pads above every ASIC cell and some embodiments will only have one contact pad, e.g., a contact pad US, formed above the active regions in some of the ASIC cells 92.

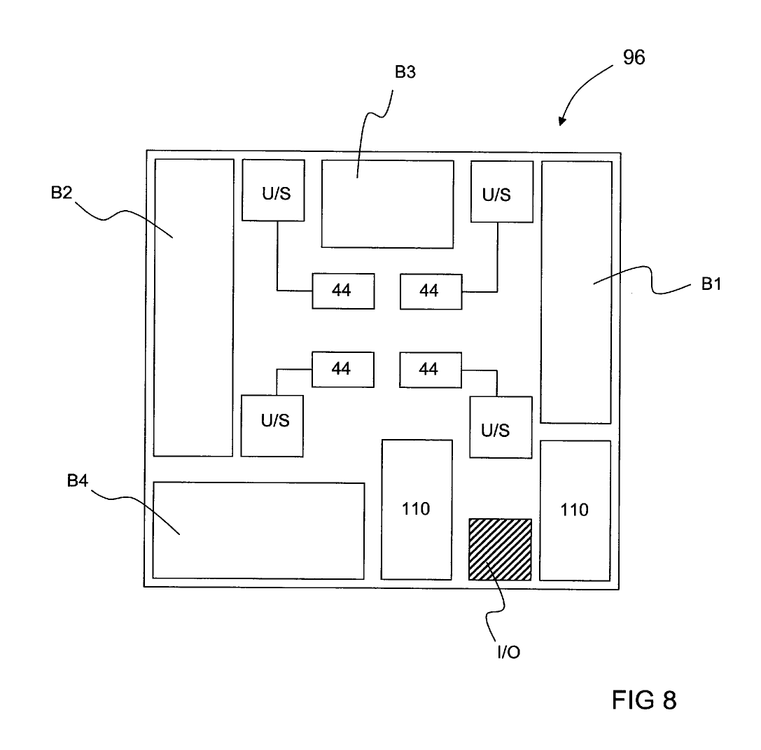

When positioning I/O circuitry and associated contact pads I/O within the array 90 there is also a need to integrate the Electrostatic Discharge (ESD) protection circuitry within the array. However, such placement of the ESD protection circuitry should not consume active area needed for circuit functions such as those shown in FIG. 4. Allocation of active area for ESD protection circuitry can be minimized by the sharing of an ESD contact pad among support circuitry for multiple, e.g., four to eight, acoustical subelements 32. Similar or identical circuit support functions performed for each of multiple individual acoustical subelements 32 can be grouped into a relatively large ASIC cell, generally referred to herein as a grouped cell. An exemplary grouped cell 96, shown in FIG. 8, provides support circuitry for a group of four acoustical subelements 32 (not shown) with improved circuit packing density and reduced signal routing. The grouped cell 96 includes four contact pads US, each for connection with a different subelement 32 to provide signals for generating an acoustic pattern during the transmit mode and for receiving echo signals during the receive mode. In addition, the grouped cell 96 includes one or more contact pads I/O which may transmit any of several signals including high voltage for the pulser circuits, ground, digital I/O and analog I/O. Other I/O signals may enter a first grouped cell 96 through additional contact pads I/O formed in metallization above the circuitry, or through contact pads I/O connected to other grouped cells wherein the signal is then routed to or from the first grouped cell 96 through interconnect in the ASIC on which the grouped cell is formed.

The grouped cell 96 includes numerous blocks Bi of circuitry including a digital block B1, and analog block B2 and a high voltage block B3 of pulser circuits 44. Circuit block B4 is representative of a layout feature wherein circuitry of a similar type, e.g., all high voltage PMOS devices, is consolidated in one area of the grouped cell 96. Forming such common regions or blocks can minimize routing and noise isolation for relatively sensitive low voltage analog circuitry in the block B2. This consolidation can effect greater area efficiency for a given level of signal isolation. In addition, ESD protection circuitry 110 is formed in the grouped cell 96 to protect circuitry associated with the signal line connected into the grouped cell through the contact pad I/O.

In the context of a grouped cell, the meaning of the term subelement remains as previously described. In the disclosed embodiments, a subelement includes an acoustical subelement 32 and its associated integrated electronics. However, for embodiments incorporating a grouped cell, the integrated electronics associated with each acoustical subelement is not formed in a separate ASIC cell specific to one acoustical subelement. Rather, such support circuitry in the form of integrated electronics is formed in combination with support circuitry associated with other acoustical subelements in one grouped cell.

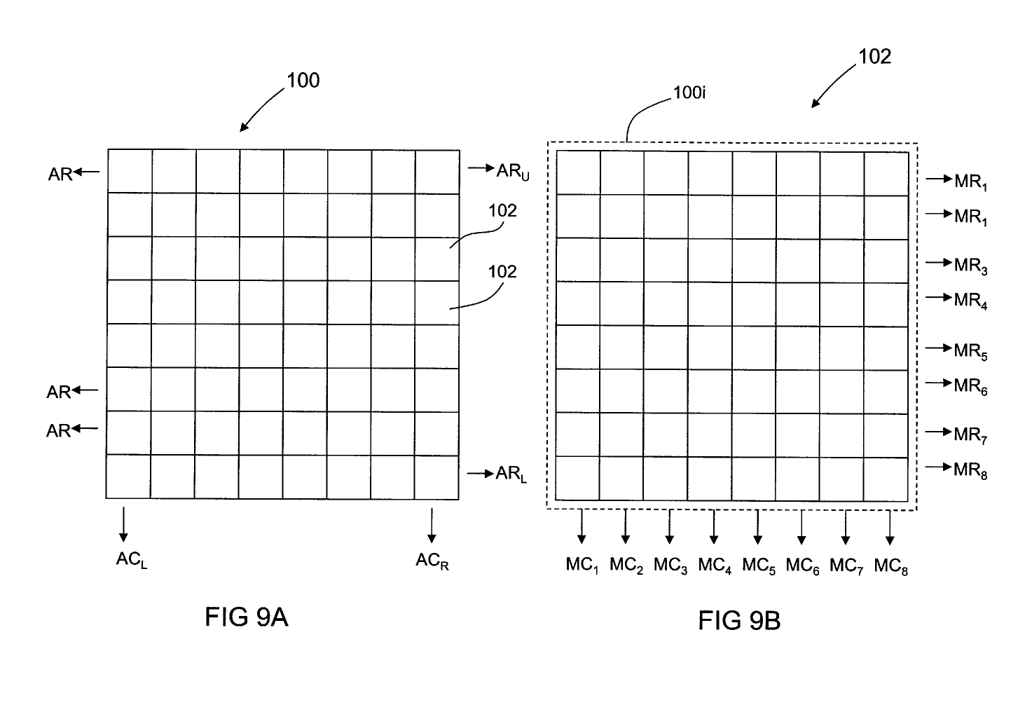

According to the invention, by forming a grouped cell to provide support circuitry to multiple acoustical subelements 32, it is possible to increase the circuit density and provide functionality not previously integrated within ASIC cells. FIGS. 9A-9B illustrate a subelement array 100 according to a configuration which can be incorporated into the ultrasound system 51. The array 100 comprises a plurality of ASICs 102 arranged in horizontal ASIC rows AR and vertical ASIC columns AC. A plurality of subelements are arranged generally according to the two dimensional array 5 of acoustical subelements 32 shown in FIG. 2, with eight acoustical subelements 32 associated with support circuitry in each ASIC 102. Multiple ones of the acoustical subelements 32 are formed in columns C of the array 5 with adjoining ASICs arranged in the columns AC so that the Columns C of acoustical subelements 32 extend along Columns AC of adjoining ASICs in a first direction (e.g., from top down in the view of the ASIC array 100 shown in FIG. 9A). The columns AC formed by adjoining ASICs in the array 100 extend along a direction orthogonal with the direction of the rows AR so that the sequence of columns C in the array 5 extends in the direction of the columns AC from ASIC to adjoining ASIC in each column. That is, with reference to FIG. 9A, the acoustical subelement columns C of FIG. 2 extend from a left-most column C positioned adjacent a left-most ASIC column AC_{L} to a right-most column C positioned along a right-most ASIC column AC_{R}. The figures show only a limited number of columns AC and rows AR in the ASIC 100 for purposes of simplified illustration, but this and other embodiments according to the invention may contain many more columns AC and rows AR of grouped cells.

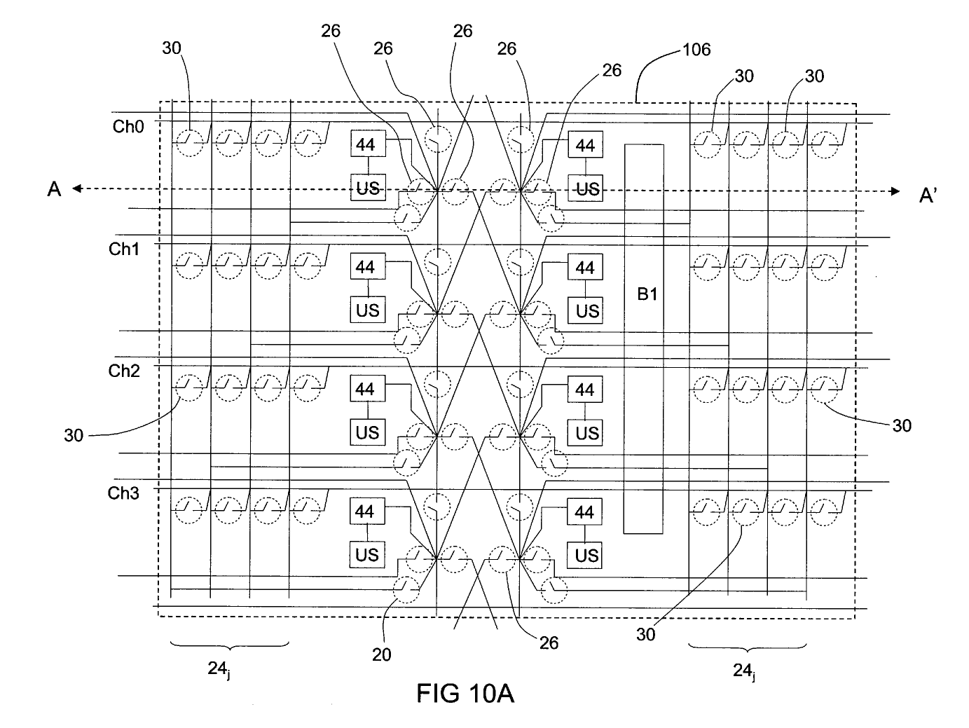

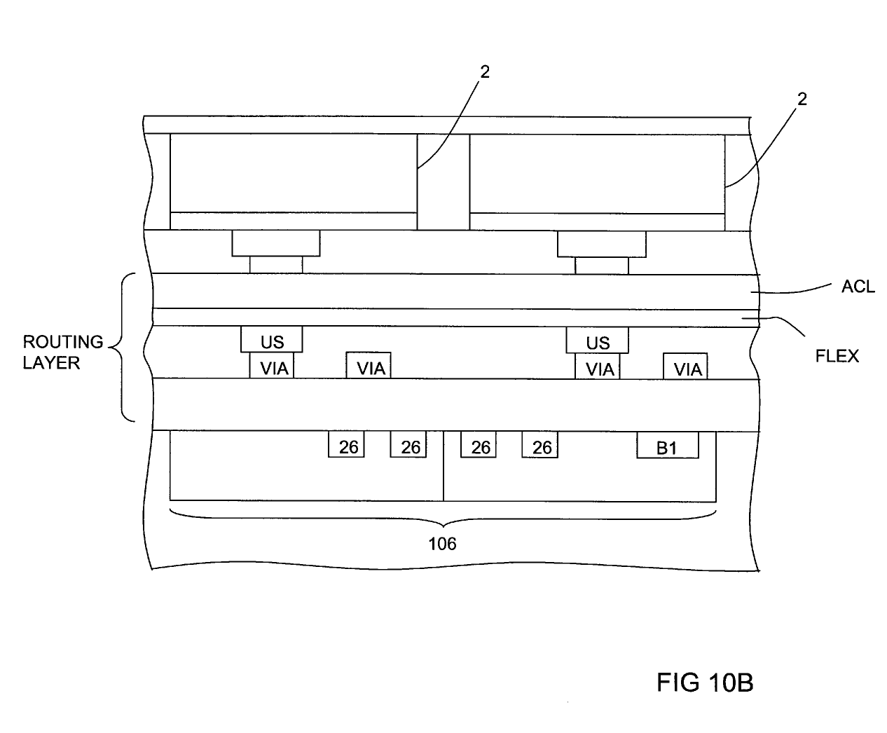

The ASICs 102, like the ASICs 88, each comprise an array of acoustical subelement support circuitry. In each ASIC 102 an array 100i of grouped cells 106 (see FIG. 10A) extend substantially throughout the ASIC including regions along the entire periphery of the ASIC. There is no requirement for a separate and distinct I/O region dedicated to placement of I/O circuitry and contact pads I/O for receiving I/O signals. As shown in FIG. 9B, the arrays 100i each include ASIC grouped cells 106 arranged in grouped cell rows MR and grouped cell columns MC with contact pads US and contact pads I/O integrated within the array 100i.

Reference is now also made to a plan view of the grouped cell 106 shown in FIG. 10A, and the associated cross sectional view of the grouped cell 106 shown in FIG. 10B. Eight contact pads US are formed in metallization above the active area of each grouped cell 106 to connect eight acoustical subelements 32 (not shown) to each of eight pulser circuits 44 in the grouped cell 106. In the simplified illustration of FIG. 9B, one hundred grouped cells 106 in the larger grouped cell array 100i are shown arranged in ten rows MR1-MR10 and ten columns CR1-CR10. One or multiple contact pads I/O may also be formed in metallization above each grouped cell 106 (as described for the grouped cell 96 of FIG. 8. With the grouped cell 106 connected to each of eight acoustical subelements 32, eight subelements are associated with the combination of one grouped cell 106 and eight acoustical subelements 32. The cross sectional view of FIG. 10B, taken along line A-A′ of FIG. 10A, illustrates an example connection pattern between a single grouped cell 106 and a pair of cMUTs which is effected with a routing layer comprising a layer of flex and associated contact pads, vias and an anisotropically conductive adhesive layer (ACAL). The contact pads US are formed in an upper portion of the ASIC metallization to connect the pulser circuits 44 through routing layers 108, which include the ASIC metallization, to the cMUTs.

As described for the grouped cell 96 of FIG. 8, the grouped cell 106 also comprises support circuitry as described in FIG. 4 for each of the associated acoustical subelements 32. The support circuitry may be distributed in a manner analogous to that described in FIG. 8 with separate blocks Bᵢ. That is, as described for the grouped cell 96, similar support circuitry associated with different acoustical subelements is consolidated into regions of common circuitry within the grouped cell instead of placing circuitry associated with each acoustical subelement in a distinct region of the grouped cell allocated to only one acoustical subelement. The subelement support circuitry is distributed in the grouped cell to optimize parameters such as area efficiency and noise isolation, to reduce the amount of routing between components and to add further circuitry into the grouped cell or reduce the size of the grouped cell relative to the size and number of acoustical subelements 32 (or groups of acoustical subelements) associated with each grouped cell. In the example illustrations, each grouped cell 106 also includes ESD protection circuitry 110 (such as shown in FIG. 8) in association with the one or more contact pads I/O bringing a signal directly into that grouped cell.

A larger number of contact pads I/O may be formed over an ASIC array 100 of grouped cells 102, relative to the number of contact pads I/O which can be formed over a region in the array 88 which contains the same number of subelements as the grouped cell but which is formed with the less area-efficient ASIC cells 92. Further, functional circuitry, including Electrostatic Discharge (ESD) protection circuitry may be placed within the grouped cell without compromising the amount of area needed for other active circuitry to support the acoustical subelements. Grouped cells can be designed to create larger, contiguous active areas that can more than compensate for the area allocated to the ESD protection structures and other circuitry such as the timing channel select switches 30_{I}.

Another feature of the grouped cells 96 and 106 is the integration of switching network components shown in FIG. 2 into the grouped cell. As shown in FIG. 10A one access switch 20 and three matrix switches 26 are associated with each pulser circuit 44. The contact pads US effect selective coupling between each acoustical subelement 32 and five other acoustical subelements.

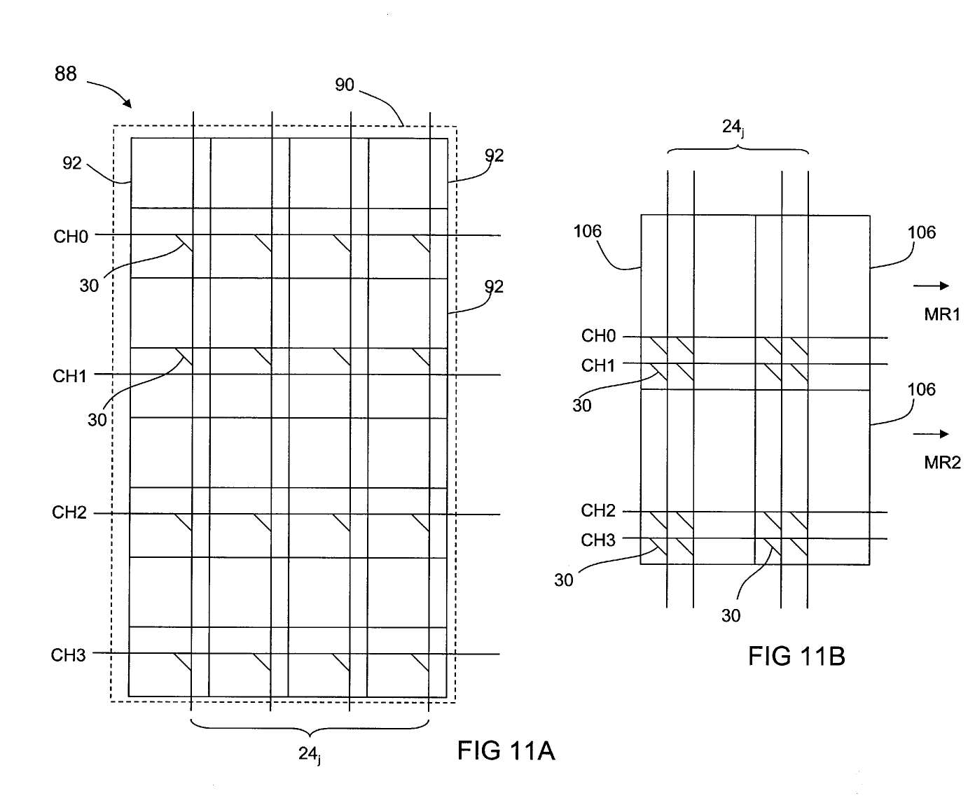

FIG. 11 provide a comparison of exemplary arrangements for routing of channel lines 28 to ASIC cells 92 of an ASIC 88 and the grouped cells 106 of an ASIC 102 to effect placement of the timing channel select switches 301. FIG. 11A shows a small group of ASIC cells 92 in a portion of the array 90 with four of the channel lines 28 (CH0, CH1, CH2, CH3) extending above the array in a level of flex circuitry. In this example, 16 timing channel select switches 30, are formed in each of 16 different ASIC cells 92. That is, for each channel line CH0, CH1, CH2, CH3, alternate ASIC cells in a row CR contain the switches 30_{I}. An access column line 24_{J} extends along each of the ASIC cell columns CC in the ASIC metallization to effect selective connection to each of the channel lines via one of the switches 30_{I}. Each channel line may extend directly into an ASIC cell containing a switch 30_{I} by connecting through a contact pad I/O formed over the active area of the ASIC cell containing the switch 30_{I}. In other embodiments, the channel lines may connect into ASIC metallization through contact pads I/O positioned on other ASIC cells 92. The access column lines 24_{J} are routed through ASIC metallization to effect connections with the switches 30_{I} in the cells 92.

FIG. 11B illustrates a small portion of a grouped cell array 100i formed along an ASIC 102. Four of the channel lines 28 (CH0, CH1, CH2, CH3) extend above the array 100i in a level of flex circuitry and along two adjoining grouped cell rows MR1 and MR2. Four timing channel select switches 30_{I} are formed in each of four adjoining grouped cells 92. In this example pairs of the channel lines (CH0, CH1), (CH2, CH3) are positioned to extend along each of the rows MR1 and MR2. Pairs of access column lines 24_{J} extend along each the two illustrated grouped cell columns MC in the ASIC metallization to effect selective connection to each of the channel lines (CH0, CH1, CH2, CH3) via one of the switches 30_{I}. Each channel line may extend directly into an ASIC cell containing a switch 30_{I}. Connection of the channel lines to the timing channel select switches 30_{I} is made through contact pads I/O formed over the active area of the grouped cells 106 containing the switch 30_{I}. In other embodiments, the channel lines may connect into ASIC metallization through contact pads I/O positioned on other grouped cells 106 to be routed to the switches 30_{I}. The access column lines 24_{J} are routed through ASIC metallization to effect connections with the switches 30, in the grouped cells 106. Additional access column lines 24_{J} may be routed along each grouped cell column MC to increase the number of access lines available to the subelement circuitry in each grouped cell. Each subelement will have at least one access switch to connect with one of the access column lines 24_{J}.

Numerous advantageous features of the invention have been described. In summary, the invention provides an architecture wherein pads conducting input-output (I/O) signals can be located within the cells of an ASIC array (e.g., cells 92 or grouped cells 96 or 106) instead of consuming area and volume outside the array. Subelements in grouped cells may share individual ESD pads. With the contact pads US and I/O distributed over the active areas of active electronic devices there is greater circuit density and a higher level of integration can be attained. The architecture permits continuous tiling of ASICs without requiring gaps in the ASIC support circuitry. This feature is achieved by moving the I/O pads and associated circuitry into the ASIC cells so that the subelement support circuitry is formed therein and no there is no need for dedicated I/O regions along the periphery of the ASICs.

Still another feature of the invention is that with the grouped cell architecture the amount of routing can be reduced with a corresponding reduction of parasitic capacitance, this leading to savings in power expended during transmission. Power economy is important for highly portable and inexpensive ultrasound systems. The area savings resulting from formation of the grouped cells enables integration of more switches and logic circuitry within the ASIC array. With the timing channel select switches formed within the exemplary ASIC cells 92 or grouped cells 96 or 106, there is a reduction in the required wiring outside of the ASIC metallization in order to effect selection of subelements to define the acoustic focusing elements. The invention enables further overall reduction in the volume required for circuitry placed in the probe unit.

While the invention has been described with reference to particular embodiments, it will be understood by those skilled in the art that various changes may be made and equivalents may be substituted for elements thereof without departing from the scope of the invention. For example, although the disclosed embodiments use cMUTs, other ultrasonic transducer technologies could be used, including PZT, pMUTs and PVDF and any future transducer technologies could also be used. In addition, many modifications may be made to adapt a particular situation to the teachings of the invention without departing from the essential scope thereof. Therefore it is intended that the invention not be limited to the particular embodiments disclosed as the best mode contemplated for carrying out this invention, but that the invention will include all embodiments falling within the scope of the appended claims.

The claimed invention is:

1. An ultrasound system comprising:
a two-dimensional array of ultrasound transducer cells grouped in a plurality of acoustical subelements, wherein transducer cells in an acoustical subelement are operable together according to a common pulser timing signal, and transducer cells in different acoustical subelements are operable according to different pulser timing signals;
an integrated circuit structure comprising an array of circuit support cells formed along a plane spaced apart from the array of ultrasound transducer cells, a first of the cells providing circuitry for implementing selectable timing signals and high voltage pulse generation to propagate acoustic signals from multiple transducer cells, including transducer cells positioned in different ones of the acoustical subelements, a plurality of connective paths extending between the transducer cells and the first of the circuit support cells to effect generation of acoustic signals from said transducer cells and receipt into the first of the circuit support cells of echo data from said multiple transducer cells, a first of the paths extending between a first transducer cell positioned in a first of said different ones of the acoustical subelements and circuitry in the first of the support cells, and a second of the paths extending between a second transducer cell positioned in a second of said different ones of the acoustical subelements and circuitry in the first of the support cells; and
a plurality of connective paths extending between the first circuit support cell and processing and control circuitry external to the integrated circuit structure to effect operation and signal processing functions in association with said multiple transducer cells.

2. The system of claim 1 wherein each of the first and second connective paths includes a contact pad formed on the integrated circuit structure and overlying the first of the circuit support cells.

3. The system of claim 1 wherein each of the connective paths extending between the first circuit support cell and processing and control circuitry external to the integrated circuit structure includes a contact pad formed on the integrated circuit structure and overlying the first of the circuit support cells.

4. The system of claim 1 wherein the first circuit support cell includes electrostatic discharge protection circuitry configured to protect circuit functions formed in other circuit support cells on the integrated circuit structure, circuitry in the first circuit support cell connected through contact pads to (i) effect electrical contact to the circuit functions protected by the electrostatic discharge protection circuitry and (ii) transmit signals received by said multiple transducer cells to processing circuitry external to the integrated circuit structure.

5. The system of claim 1 wherein the first of the circuit support cells is configured to provide pulse generation to propagate acoustic signals from the transducer cells in each of at least four subelements.

6. The system of claim 5 wherein the first of the circuit support cells includes a different pulser circuit for each of the at least four acoustical subelements for providing the pulse generation to propagate acoustic signals from the transducer cells.

7. The system of claim 5 wherein the first of the circuit support cells is configured to provide pulse generation to propagate acoustic signals from the transducer cells in each of at least eight acoustical subelements.

8. The system of claim 1 including ESD protection circuitry formed in the first of the circuit support cells to protect circuitry connected to the first of said different ones of the acoustical subelements and the second of said different ones of the acoustical subelements.

9. The system of claim 1 wherein all of the circuit support cells are configured to provide pulse generation to propagate acoustic signals from the transducer cells in each of at least four acoustical subelements and each circuit support cell includes a different pulser circuit for each of the at least four acoustical subelements for providing the pulse generation to propagate acoustic signals from the transducer cells.

10. The ultrasound system of claim 1 further including:
a plurality of additional integrated circuit structures each also comprising an array of circuit support cells formed along a plane spaced apart from the array of ultrasound transducer cells, with a first of the cells providing circuitry for implementing selectable timing signals and high voltage pulse generation to propagate acoustic signals from multiple transducer cells, including transducer cells positioned in different ones of the acoustical subelements, all of the acoustical subelements and intergrated circuit structures assembled in a probe unit.

11. The ultrasound system of claim 10 further including an electronics unit separate from the probe unit including the processing and control circuitry.

12. The ultrasound unit of claim 11 wherein the electronics unit is coupled to provide signals to the probe unit via a cable.

13. A method of configuring an ultrasound system comprising:
providing a two-dimensional array of ultrasound transducer cells grouped in a plurality of acoustical subelements, wherein transducer cells in an acoustical subelement are operable together according to a common pulser timing signal, and transducer cells in different acoustical subelements are operable according to different pulser timing signals;
positioning an integrated circuit structure along a plane spaced apart from the array of ultrasound transducer cells, the integrated circuit structure comprising an array of circuit support cells;
providing circuitry in a first of the cells for implementing selectable timing signals and high voltage pulse generation to propagate acoustic signals from multiple transducer cells, including transducer cells positioned in different ones of the acoustical subelements;
providing a plurality of connective paths extending between the transducer cells and the first of the circuit support cells to effect generation of acoustic signals from said transducer cells and receipt into the first of the circuit support cells of echo data from said multiple transducer cells, with a first of the paths extending between a first transducer cell positioned in a first of said different ones of the acoustical subelements and with a second of the paths extending between a second transducer cell positioned in a second of said different ones of the acoustical subelements; and
providing a plurality of connective paths extending between the first circuit support cell and processing and control circuitry external to the integrated circuit structure to effect operation and signal processing functions in association with said multiple transducer cells.

14. The method of claim 12 further including forming a contact pad on the integrated circuit structure along the first and second connective paths and overlying the first of the circuit support cells.

15. The method of claim 13 further including forming electrostatic discharge protection circuitry in the first circuit support cell to protect circuit functions formed in other circuit support cells on the integrated circuit structure.

16. The method of claim 15 further including connecting circuitry in the first circuit support cell through contact pads to (i) effect electrical contact to the circuit functions protected by the electrostatic discharge protection circuitry and (ii) transmit signals received by said multiple transducer cells to processing circuitry external to the integrated circuit structure.

17. The system of claim 13 further including configuring the first of the circuit support cells to provide pulse generation to propagate acoustic signals from the transducer cells in each of at least four subelements.

18. The system of claim 17 wherein the step of configuring to provide pulse generation includes providing a different pulser circuit for each of the at least four acoustical subelements for providing the pulse generation to propagate acoustic signals from the transducer cells.
